(19) United States
(12) Patent Application Publication
(10) Pub. No.: US 2025/0226081 A1
(43) Pub. Date: Jul. 10, 2025

(54) METHOD FOR INTEGRATING AND EXECUTING AN APPLICATION IMPLEMENTED BY A MEDICAL IMAGING PLATFORM

(71) Applicant: OLEA MEDICAL, LA CIOTAT (FR)

(72) Inventors: Bruno LOPEZ, La Ciotat (FR); Philippe PETER, Le Beausset (FR)

(73) Assignee: OLEA MEDICAL, LA CIOTAT (FR)

(21) Appl. No.: 18/853,579

(22) PCT Filed: Apr. 3, 2023
(86) PCT No.: PCT/EP2023/058715

§ 371 Date: Oct. 2, 2024

(30) Foreign Application Priority Data
Apr. 4, 2022 (FR) FR2203056

(51) Int. Cl. G16H 30/20; G06F 8/65

(52) U.S. Cl. CPC G16H 30/20; G06F 8/65

(57) ABSTRACT

The invention relates to a method for integrating and executing a software application to be implemented by a medical imaging platform running a file system. Such a method comprises a step of reading an agreement dedicated to a software application, the agreement comprising the access paths for input and/or output directories of the file system in order to write and/or read the values of the input/output data of the software application. The invention further relates to a medical imaging platform suitable for implementing such a method for integrating and executing software applications.

The invention relates to a method for integrating and executing an application for analysing medical images implemented by a medical imaging platform.

It is known to use a medical imaging platform to assist a practitioner to identify pathologies by using medical images optionally acquired by different methods, for example by means of a magnetic resonance imaging (MRI) appliance. Such a medical imaging platform is designed to host one or more software application(s) that is/are generally specialized for identifying a given pathology (stroke, pneumonia, tumour, etc.), analyzing a specific organ (brain, lung, breast, etc.) or estimating specific physiological or haemodynamic parameters (tissue density, blood flows or volumes, etc.). In a hospital environment, the medical images are generated from one or more medical imaging appliances (MRI, scanners, etc.) and are transmitted to said platforms or stored within picture archiving and communication (PACS) servers. Moreover, the medical images are encoded and transmitted according to a specific protocol known as digital imaging and communications in medicine (DICOM), in order to facilitate transfers of medical images between different manufacturers' imaging appliances and systems.

Thus a medical imaging platform hosts a plurality of software applications for processing a plurality of cases (in terms of pathologies, organs, acquisition methods). The DICOM format makes it possible to associate additional meta-information with the images per se, on the basis of which a medical imaging platform “decides” to trigger execution of one or other software application. Thus, the document US2019/0108905A1 describes an embodiment according to which, as a function of said items of DICOM meta-information, a configuration file of a data processing pipeline implementing several software applications (called filters) installed on a platform, can be selected from a plurality of configuration files, or can be created, to define the data processing process for such medical images.

However, the currently available platforms cannot include at all times all the software applications to fit a constantly increasing diversity of cases nor the most up-to-date versions of said software applications in order to keep benefitting from the many innovations in this field. It is therefore necessary to enrich or update the plurality of software applications within the medical imaging platforms deployed on the sites.

From a technical point of view, a medical imaging platform is similar to a computer including a processor unit consisting of one or more microprocessors or microcontrollers implementing program instructions of a hardware operating system (data or program memories, peripherals, etc.) and hosted software applications. Said program instructions are loaded in one or more memories of said platform in communication with said processor unit.

By “memory or storage means” is meant any computer memory, whether volatile or not. A non-volatile memory is a computer memory the technology of which makes it possible to retain its data in the absence of an electrical power supply. It can contain data resulting from inputs, calculations, measurements and/or program instructions. The main non-volatile memories currently available are of the type capable of being written to electrically, such as EPROM (erasable programmable read-only memory) or also written to and erased electrically, such as EEPROM (electrically erasable programmable read-only memory), flash, SSD (solid-state drive), etc. Non-volatile memories are distinguished from the memories known as “volatile”, from which the data are lost in the absence of an electrical power supply. The main types of volatile storage currently available are RAM type (random access memory, also named “read-write memory”), DRAM (dynamic random-access memory, needing to be regularly refreshed), SRAM (static random-access memory needing to be refreshed in this way when there is a loss of electrical power), DPRAM or VRAM (particularly suitable for video), etc.

The known medical imaging platforms have, or require, methods for software application integration. The most widespread integration method uses the DICOM standard. According to this method, each software application is designed to process input data in the DICOM format only and to deliver results in this same format only, said data moreover must conform to a specific communication protocol. However, this first integration method has major drawbacks. Firstly, the DICOM format and protocol are not optimum for generating and sharing a report based on the intermediate results generated by other software applications. Moreover, the DICOM standard does not make it possible to easily configure software applications according to certain contexts of reception and/or exploitation of the imaging data to be analyzed.

Other known platforms have opted for a second integration method based on using application programming interfaces (API). This solution is advantageous as it facilitates access to configuration services for said software applications and offers numerous possibilities for exporting the results. On the other hand, this second method generally has a major drawback resulting from the fact that an API is generally specific or dedicated to a given medical imaging platform or a given manufacturer of such platforms. In order to be able to develop an application capable of being integrated into a first medical imaging platform, the designer of the application must know the API required by said first platform and construct their application around this API. If said application is to be integrated in a second platform requiring a second API different from the first, said designer must modify the design of their application and produce a second software application. Moreover, it is difficult or even impossible for said designer to test the correct operation of an application before its deployment in the absence of a test platform provided for their use. For this purpose they must have available an emulator of said platform or a stub for simulating the behaviour thereof, which is not always the case, or can prove complex or costly.

At most, an applications developer can give a certain independence to the “business” code of an application with respect to the format of the data handled by virtue of generating a description of said data, transmitted concomitantly with said data handled, as indicated in the document “Data description for data independence” by W. C. McGee, a technique better known today as “hexagonal architecture”. However, such a teaching does not provide any solution conferring independence on said application with respect to the host platforms.

The invention overcomes the drawbacks arising from the state of the art.

Among the many advantages achieved by the invention, there may be mentioned more specifically that the invention makes it possible to design software applications independently of a particular medical imaging platform. By virtue of the invention, said software applications can exploit and/or generate data that conform to the DICOM standard or according to any other format. Thus, the software applications are integrated in a medical imaging platform in the form of autonomous application modules specifying their dependencies, the inputs and outputs of which are read or written on the file system used by any medical imaging platform. The invention thus achieves very high interoperability, no longer linking an application to a given type of platform as is still the case today. Moreover, a designer of an application has the ability to very easily test the operation of their application before its deployment, without the need for acquisition or immobilization of a medical imaging platform for debugging purposes, a simulator/emulator thereof or even, as a variant, a sophisticated stub, too limited to simulate all of the functionalities of such a platform.

In the remainder of the document, by “file system” is meant the services operated by a medical imaging platform or by any computer, which determine the organisation of the read- or write-accessible data within a memory, or more generally within a physical or logical volume of such a memory. As standard, such a file system determines the way in which said data are stored and organizes them in logical structures known as files. Such a file system makes it possible to process, store and share data between several computer programs such as an operating system of the computer resources (or in this case a medical imaging platform) and the software applications that call on said resources by means of said operating system. A file system thus provides an abstract view of the data stored in a memory and makes read- and/or write-access thereto possible from an access route known as a path.

For this purpose, the invention provides a method for integrating and executing a software application, said method being designed to be implemented by a medical imaging platform operating a file system. Such a 15 method includes:
a step of reading a contract dedicated to a software application that generates an item of output data based on an item of input data, said contract consisting of an item of digital content including:
   a unique identifier denoting said software application;
   an input virtual directory intended to contain a value of the item of input data of the software application;
   an output virtual directory intended to contain a value of the item of output data of the software application;
a step of writing the value of the item of input data of the software application to an input directory of the file system;
a step of triggering execution of the software application denoted by the unique identifier with as arguments the access paths to the input directory of the file system and to an output directory of said file system, said access paths being respectively associated with the input and output virtual directories read from said contract;
a step of reading the value of the item of output data of the software application from the output directory of the file system.

In order to make it possible to easily parameterize the execution of a software application, the contract can include a value of an operating parameter of said software application. In this case, the step of triggering execution of said software application of a method according to the invention can be adapted to include as argument the value of said operating parameter.

In order to realize the integration of a software application, a method according to the invention can comprise a step of loading the software application and the contract in a memory of the software platform.

Advantageously, and in particular to facilitate prior testing of a software application, the latter can adopt the form of a computer container, the unique identifier denoting said software application comprised in said contract expressing the image of said computer container.

Advantageously, the input virtual directory intended to contain the item of input data of the software application can be associated in the contract with an attribute specifying the optional or mandatory nature of said item of input data. In this case, when such an attribute shows the optional nature of said item of input data, it is possible for the step of writing the latter to be implemented only if such an item of input data is available for the medical imaging platform.

The invention also provides for enriching with meta-information an item of output data generated by a software application integrated and executed according to the invention. For this purpose, the output virtual directory intended to contain the value of the item of output data of the software application can be associated in the contract with a typing attribute of said output. The step of reading the value of the item of output data of the software application can then consist of associating said value of said typing attribute with the value of said item of output data.

To provide safeguarding against any malicious change to a contract associated with a software application, said contract can be encoded prior to its integration in the platform, or after any update thereof by said platform. In this case, the step of reading a contract can include a prior sub-step of decoding said contract.

According to a second purpose, the invention relates to a computer program product including program instructions that can be executed by the processor unit of a computer, said program instructions being capable of being loaded in a non-volatile memory of said computer and the execution of which by said processor unit causes the implementation of a process integrating and executing a software application according to the invention.

According to a third purpose, the invention relates moreover to a computer-readable storage medium including the instructions of such a computer program product.

Finally, the invention relates moreover to a medical imaging platform comprising a processor unit, a memory used by a file system, said memory comprising the program instructions of a computer program product according to the invention.

Other characteristics and advantages will become more clearly apparent on reading the following description and examination of the accompanying figures in which:

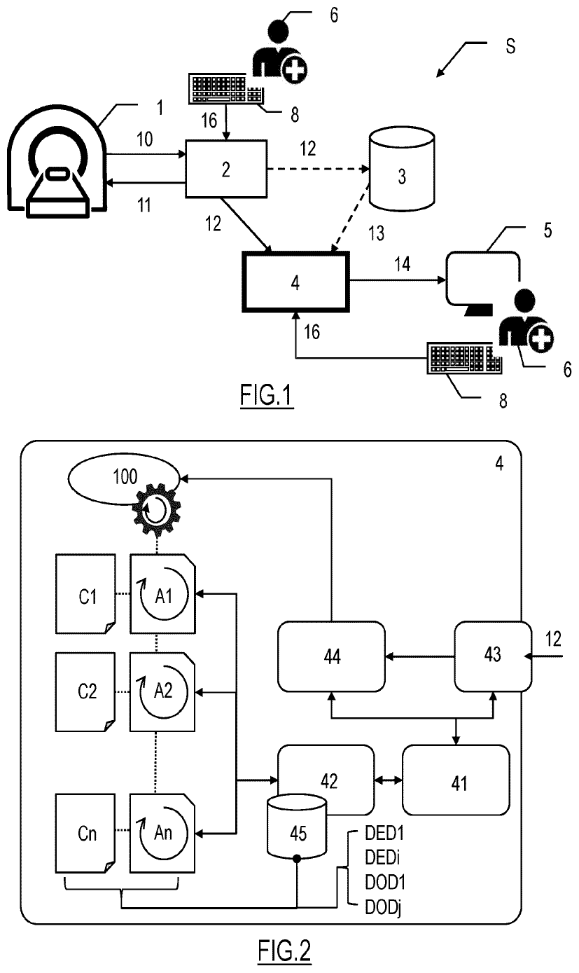

FIG. 1 shows a medical imaging system comprising a medical imaging platform;

FIG. 2 shows the simplified architecture of a medical imaging platform according to the invention;

FIG. 3 shows an example design of a software application that can be integrated and executed by a medical imaging platform according to the invention;

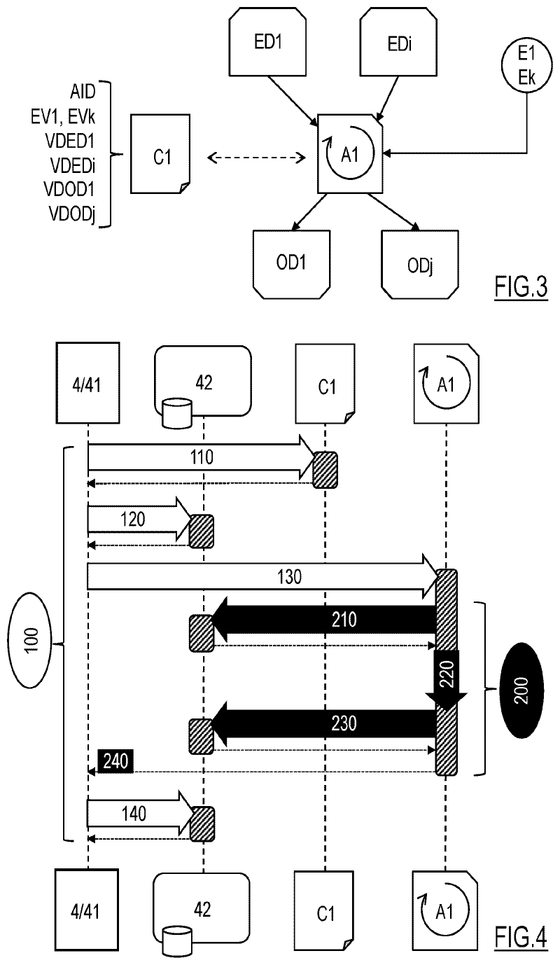

FIG. 4 shows a method for integrating and executing a software application by a medical imaging platform according to the invention.

FIG. 1 shows an example medical imaging system S comprising an imaging appliance 1. The latter delivers a plurality of digital image sequences 12 of one or more parts of the body of a patient, by way of non-limitative example, the brain, the heart, the lungs. The magnetic resonance imaging appliance 1 is generally controlled using a console 2. A user 6, for example an operator, practitioner or researcher, can thus choose commands 11 for controlling the imaging appliance 1, from parameters or instructions 16 entered via a human-machine input interface 8 of the analysis system S. Such a human-machine interface 8 can consist for example of a computer keyboard, a pointing device, a touchscreen, a microphone or, more generally, any interface arranged to translate a gesture or an instruction given by a human 6 into data controlling or parameterizing an imaging appliance 1. On the basis of items of information 10 generated by said appliance 1, a plurality of digital image sequences 12 is obtained of a part of a body of a human being or an animal.

The image sequences 12 can optionally be stored within a server 3 or PACS, i.e. a computer equipped with its own storage means, and constitute a patient's medical file 13. Such a file 13 can comprise images of different types, such as functional images demonstrating the activity of the tissues, or anatomical images reflecting the properties of the tissues. The image sequences 12 or, more generally, the experimental data, are analyzed by a medical imaging platform 4 arranged for this purpose in order to generate text or graphic indicators 14 that can be visualized by means of a human-machine output interface 5 by health care personnel 6 according to instructions 16 sent to said medical imaging platform 4.

FIG. 2 depicts an example architecture of a medical imaging platform 4 according to the invention and intended to be integrated in an imaging analysis system such as the system S shown in FIG. 1. Such a medical imaging platform 4 can include a processor unit in the form of one or more microprocessors or microcontrollers 41 implementing appropriate application program instructions loaded in storage or memory means 45 of said imaging analysis system S. Read- and/or write-access to said memory 45 by the processor unit 41 can be managed by a file system 42. Said processor unit 41 is advantageously in communication with an input/output module 43 responsible in particular for receiving medical image sequences 12, or with a module 44 responsible for determining, based on said image sequences 12 received, which software application or applications among the available software applications A1, A2, An, merit implementation for analyzing said image sequences 12 with the aim of generating relevant quantities of interest 14 intended for the health care personnel 6. Said software applications A1 to An, or more precisely the program instructions that characterize them, are loaded beforehand in memory 45 of the platform 4. As described below, each software application A1, A2, An is associated with a distinct item of digital content of said software application that is dedicated thereto, an item of digital content that will be referred to hereinafter as “contract” C1, C2, Cn. Thus, the program instructions of the software application A1, with a contract C1 in the form of an item of digital content including items of information that can be exploited or interpreted by the processor unit 41, are loaded together, more specifically according to a method 100 for integrating and executing software applications according to the invention. In order to adapt the operation of the medical imaging platform 4, a computer program including the instructions that can be executed by the processor unit 41 of said platform 4 is loaded in the memory 45, said program instructions causing implementation of such an integration and execution method 100.

FIG. 3 shows an example functional description of a software application according to the invention. Such a software application consists of digital processing bearing on one or more items of input data to generate one or more items of output data. By way of example, such a first software application can consist of an application aiming to categorize a stream of DICOM images 12 acquired by an X-ray imaging appliance and generate items of output data sorted by type, said images being enriched by one or more labels intended to classify said images. Such labels can be selected from a set of acronyms “CTP” (computed tomography perfusion), “CTA” (computed tomography angiography), “NCCT” (non-contrast computed tomography) describing acquisition methods. In a variant, such a first software application could use items of input data in the form of DICOM images labelled “CTP” and generate at output items of output data in the form of charts showing a haemodynamic parameter and a results report expressed in a text form and intended ultimately to be sent by email. A second software application could be designed to use items of input data in the form of a stream of DICOM images 12 acquired from a magnetic resonance imaging appliance and generate items of output data sorted by type, said images being enriched by one or more labels intended to classify said images. Such labels could be selected from a set of acronyms DWI (diffusion-weighted imaging), PWI (perfusion-weighted imaging), FLAIR (fluid-attenuated inversion recovery) describing acquisition methods of the imaging appliance.

Generally, and as shown in FIG. 3, a software application A1 generates items of output data OD1, . . . , ODj based on items of input data ED1, . . . , EDi. Said items of input and/or output data respectively can have different formats, for example text, DICOM or other types. A software application A1 can moreover exploit one or more environment variables E1, . . . , Ek. Such environment variables are transmitted to the software application during its execution, with the aim of parameterizing said execution of the software application or the processing implemented thereby. According to a preferred embodiment, the application A1 can be defined in the form of a computer container the image of which adopts the form of a static file containing executable code (i.e. a set of program instructions) and defining the parameters or dependencies necessary for its execution. Such a technical choice of “containerization” of the software applications makes it possible in particular to simplify the step of testing the operation of a software application before its deployment but without thereby requiring a test configuration similar to a medical imaging platform. In fact, the pattern of the software application is clearly defined, the container image can be implemented and tested like an autonomous application module, in the manner of a light, modular virtual machine. Taking the example of the Docker technology initially designed for processing Linux containers (Linux is a family of open-source operating systems of the Unix (AT&T) type created by Linus Torvalds). Let the identifier AID of the image of the software application A1 shown in FIG. 3 denote version 1.0 of the software application “my_application”. Execution of said software application A1 by a computer could be caused by manually invoking the command:

| | |
|---|---|
| “ docker run | \ |
| - v /tmp/inputs/ED1:/inputs/ED1 | \ (arg1) |
| - v /tmp/inputs/ED1:/inputs/EDi | \ (arg2) |
| - v /tmp/outputs/OD1:/outputs/OD1 | \ (arg3) |
| - v /tmp/outputs/ODj:/outputs/ODj | \ (arg4) |
| - e E1=VALUE1 | \ (arg5) |
| - e Ek=VALUEk | \ (arg6) |
| my_application:1.0 ” | \ (arg7) |

The command argument (arg1) makes it possible to define or resolve a link (operation also known as “mapping”) between the “physical” location or directory DED1 of the item of input data ED1 (in this case according to the example shown in FIG. 2 a temporary directory of the file system 45 of a medical imaging platform 4 or more generally of a development or test computer) said directory DED1 being accessible via the access path DPED1 “/tmp/inputs/ED1” and the “virtual” directory VDED1 (in this case, in the example shown in FIG. 3, the virtual directory “/inputs/ED1”) defined by the designer of the application A1 in the application module. Thus, when said software application A1 is executed, it reads the value of the item of input data ED1 from the virtual directory VDED1 “/inputs/ED1” while physically, i.e. in the memory 45 of the machine 4 implementing said software application A1, said virtual directory corresponds to a physical directory DED1 selected by said user, in this case the directory for which the access path DPED1 is “/tmp/inputs/ED1”.

Similarly, the arguments (arg2), (arg3) and (arg4) respectively define, for the items of data EDi, OD1 and ODj, the links between the virtual directories VDEDi “/inputs/EDi”, VDOD1 “/outputs/OD1”, VDODj “/outputs/ODj” and the physical directories DEDi “/tmp/inputs/EDi”, DOD1 “/outputs/OD1” and DODj “/outputs/ODj”.

The arguments (arg5) and (arg6) make it possible to transmit to the application A1 the values VALUE1 and VALUEk respectively to the environment variables E1 and Ek.

Finally the command argument (arg7) denotes the identifier AID of the image of the application A1, in this case “my_application” version 1.0.

The invention should not be considered limited to this choice alone of implementation of a software application in the form of a container or “Docker” technology. In a variant, it could rely on an equivalent solution such as the “podman” solution developed by Red Hat. A software application could also consist of a “standard” executable program instead of a container. In all cases, in order to be run by a method for integrating and executing software applications according to the invention such as the method 100 an example implementation of which is shown in FIG. 4, a software application such as the application A1 described in FIG. 2 is associated with a distinct item of digital content C1, known as a contract. By running this, for example by the module 44 shown in FIG. 2, execution of a software application can be triggered by unambiguously referencing it, by resolving the links between the physical locations DED1, DEDi of the items of input data ED1, EDi, the physical locations DOD1, DODj of the items of output data OD2, ODj on the imaging platform 4 and the virtual or “internal” locations VDED1, VDEDi, VDOD1, VDODj in the application module, or by defining values for environment variables E1 to Ek. The joint running of this contract C1 and the file system 42 makes it possible to facilitate integrating and executing a software application.

Thus, with reference to FIG. 2 and FIG. 3, a contract C1 includes a set of fields or elements that can be distinguished from one another for example by tags or any other equivalent technical means, so that prior to execution per se of a software application optionally parameterized by environment variables E1, Ek, the values of the items of input data ED1, EDi are written to memory locations 45 by means of the file system 42 and at the end of execution of said software application A1, the items of output data OD1 and ODj generated thereby can be used by said platform 4. The contract C1, associated with the software application A1, can advantageously comprise:
the unique identifier AID of the software application A1, in this case in FIG. 3, the container image;
the values EV1 and EVk to be respectively assigned to the environment variables E1 and Ek during execution of said software application A1;
the input virtual directories VDED1, VDEDi intended to contain the values of the items of input data ED1, EDi of the software application A1;
the output virtual directories VDOD1, VDODj intended to contain the values of the items of output data OD1, ODj of the software application A1.

More generally, such a contract C1 includes per item of input ED1, EDi or output OD1, ODj data, the virtual locations VDED1, VDEDi, VDOD1, VDODj for writing/reading a value of said item of input ED1, EDi or output OD1, ODj data. The presence of a value EV1, EVk to be assigned to an environment variable is optional, as certain software applications do not require such operational parameters as, for example, for selecting a colour palette from a plurality or a repository of units. The format of a contract is imposed by the medical imaging platform so that the latter can have the ability to use (i.e. to read and optionally to update) said contract C1. On the other hand, the content of said contract C1 is imposed by the software application according to the items of input data, outputs/environment variables that it requires. After installing the software application and its contract in memory 45 of the medical imaging platform 4, it is the task of the platform operator or the platform per se to modify and/or to parameterize said contract C1 for example by entering the physical access paths DPED1, DPDEi, DPOD1, DPODj to the directories DED1, DEDi, DOD1, DODj of the file system 42 managing the memory 45 of said platform 4 for storing the items of input ED1, EDi and output OD1, ODj data of said application A1 if said input DED1, DEDi and output DOD1, DODj directories are immutable, as well as to set the values of environment variables EV1, EVk if these exist or are required. Preferably, said access paths DPED1, DPDEi, DPOD1, DPODj are not entered in the contract C1 so as to leave to the imaging platform 4 the task of allocating temporary directories DED1, DEDi, DOD1, DODj prior to the execution per se of the application A1. Resolving the links between the virtual directories VDE1, VDEi, VDO1, VDOj and the physical directories DED1, DEDi, DOD1, DODj is performed by the arguments of the execution command, as shown above with reference to the application “my_application”. Mention may be made of a configuration step of the software application A1 at the end of its installation or loading in memory 45, for qualifying said optional update of the contract C1.

FIG. 4 shows the implementation and the functional description of a method 100 for integrating and executing a software application according to the invention. Such a method 100 is implemented by a medical imaging platform such as the platform 4 according to FIG. 1 and FIG. 2. The operation of the latter is adapted by introducing into the memory 45 a computer program the program instructions of which, during their execution by the processor unit 41 optionally under the control of a decision module 44, cause the implementation of such a method 100. It can be envisaged for the memory 45 to include moreover the software application A1, for example in the form of a container associated with a contract C1, the latter adopting the form of a structured text file, i.e. arranged by means of appropriate tags, in the manner of the software application A1 and the contract C1 already described with reference to FIG. 3.

Receiving a medical image sequence 12 used by the processor unit 41 triggers the implementation of the method 100 in order to cause execution of the software application A1 deemed appropriate.

The main steps of said method 100 are shown by white solid arrows in FIG. 4. Furthermore, the processing steps pertaining to the software application A1 are shown by black solid arrows in said FIG. 4. The latter moreover depicts the interactions between the main “actors” concerned by implementation of said method 100 (i.e. the processor unit 41 of platform 4, the file system 42 operated thereby, the contract C1 and the software application A1).

Said method 100 includes a first step 110 of reading from the memory 45 the contract C1 dedicated to the software application A1. By way of reminder, the latter generates two items of output data OD1 and OD2 based on items of input data ED1 and ED2. When the contract C1 includes access paths DPED1, DPEDi to directories DED1, DEDi of the file system 42 that must be run to initialize respectively values of the items of input data ED1 and EDi, the processor unit 41 recognizes in response (symbolized by an arrow with a fine dashed line) said access paths DPED1, DPEDi. Similarly, when the contract C1 includes access paths DPOD1, DPODj to directories DOD1, DODj of the file system 42 that must be run to read respectively values of the items of output data OD1 and ODj that will be generated by the software application A1, the processor unit 41 recognizes said access paths DPOD1, DPODj. Said processor unit 41 can moreover also recognize the environment variables E1, Ek optionally run by the software application A1 the values EV1, EVk of which will be transmitted during execution per se of said software application A1.

In a variant, according to a more general case, said contract C1 has not been subjected to modification or configuration by the medical imaging platform 4. The step 110 of the method 100 moreover includes a sub-step (not shown in FIG. 4 for the sake of simplicity) of allocating or determining temporary directories DED1, DEDi of the file system 42 that must be run to initialize respectively values of the items of input data ED1 and EDi and of the directories DOD1, DODj of the file system 42 that must be run to read respectively values of the items of output data OD1 and ODj.

The method 100 now includes a step 120 of writing the value of each item of input data ED1, EDi of the software application A1 to the input directory DED1, DEDi of the file system 42 as specified by said contract C1 or determined by the medical imaging platform 4.

Said method 100 includes a step 130 of initializing execution of the software application A1 the image of which is denoted by the unique identifier AID taken from step 110. Such a step 130 is accompanied by optionally transmitting values of environment variables when the software application so requires, in this case the values EV1 and EVk of the variables E1 and Ek. To resolve the links between the physical input DED1, DEDi and output DOD1, DODj directories (i.e. those for which the read/write access is managed by the file system) and the input VDED1, VDEDi and output VDOD1, VDODj virtual directories, the step 130 consists moreover of transmitting the access paths DPED1, DPEDi, DPOD1, DPODj to said input DED1, DEDi and output DOD1, DODj directories for each item of input ED1, EDi and output OD1, ODj data in question. This step 130 thus consists of automatically constituting an equivalent of the manual command mentioned above with reference to the test of the software application “my_application” in the form of a container, the arguments necessary for execution of the application being transmitted to said software application according to the requirements (elements, tags and/or order) specified by the contract C1.

The method 100 awaits the result of execution of the software application A1 (by means of an execution report 240 generated thereby, symbolized by an arrow with a fine dashed line in FIG. 4). In fact, at the end of step 130, the processor unit 41 implements the application A1, which includes in turn a first step 210 intended to read the values of the items of input data ED1 and EDi from the directories DED1, DEDi of the file system 42, then implements processing 220 bearing on said items of input data ED1, EDi to generate items of output data OD1, ODj. As the links between the physical (memory 45/file system 42) and virtual directories have been resolved beforehand by the execution arguments transmitted in step 130 according to said contract C1, the values of said items of output data are entered by the software application A1 in a step 230 into the directories DOD1, DODj of the file system 42. Implementation of the software application A1 ends with a step 240 of generating an execution report specifying for example if said execution was performed with or without error.

A method 100 according to the invention now includes a step 140 of reading the value of each item of output data OD1, ODj of the software application A1 from the output directory DOD1, DODj of the file system 42 as defined by the contract C1 or by the platform 4.

At the end of implementation of the method for integrating and executing the software application A1, the medical imaging platform 4 can trigger implementation of subsequent processing intended to use the result generated by said application A1. Such a subsequent processing can consist of a new instance of said method 100 causing execution of a second software application A2, An.

Advantageously, the invention provides for a software application A1, A2, An to be able to manage an item of input data or an item of output data having an optional nature. For this purpose, the optional nature can be specified in the contract C1, C2, Cn that is associated with said application A1, A2, An. According to a preferred embodiment, with reference to the application A1 shown in FIG. 3, the field or element of said contract C1 determining the virtual directory VDED1, VDEDi intended to contain an item of input data ED1, EDi can be associated with an attribute specifying an optional or mandatory nature of said item of input data ED1, EDi. According to this embodiment, a method 100 according to the invention is adapted such that, when such an attribute shows an optional nature of an item of input data ED1, EDi, the step 120 of writing the value thereof is implemented only if such an item of input data ED1, EDi is available for the medical imaging platform 4. Initializing execution of the software application A1 will not raise an error in the absence of an argument dedicated to such an optional item of data in the command 130. Such an attribute can thus consist of a Boolean value for characterizing such an optional or mandatory nature of an item of input data. In a variant, the absence of such an attribute can signify that said item of input data is mandatory in nature.

The same applies for an item of output data OD1, ODj that can be optional according to the result generated by a software application such as the application A1 according to FIG. 3. According to this embodiment, the field or element of said contract C1 determining the virtual directory VDOD1, VDODj intended to contain an item of output data OD1, ODi can be associated with an attribute specifying the optional or mandatory nature of said item of output data OD1, ODj. When such an attribute shows an optional nature of said item of output data OD1, ODj, the latter can include a determined value specifying a disregard value. In this way, the processor unit 41 knows that such an item of output data is to be disregarded if said item of data denotes a value equal to said determined value.

Furthermore, the invention provides for adding one or more items of meta-information, such as a label or a tag or, more generally, a typing attribute for classifying an item of input ED1, EDi and/or output OD1, ODj data of a software application such as the application A1 according to FIG. 3. When such an item of output data OD1, ODj can be enriched or classified by the presence of a label, the contract C1 associated with said software application A1 can be arranged so that the output virtual directory VDOD1, VDODj intended to contain the value of the item of output data can be associated with such a typing attribute of said item of output data OD1, ODj. In this case, the step 140 of reading (from the method 100 according to FIG. 4) the value of the item of output data OD1, ODj in question can consist of associating said value of said typing attribute with the value of said item of output data. When the software application A1 does not provide for such a typing, the contract C1 that is associated therewith does not associate such a typing attribute. On the other hand, the imaging platform 4 can update said contract C1 to add said typing attribute to the output virtual directory of the item of output data in question. Such enrichment of an item of output data generated by a software application can thus be exploited by the medical imaging platform 4 for selecting items of input data ED1, EDi from the software application A1 to enter into the input directory DED1, DEDi of the file system 42 or to select input data for any other application.

The invention provides moreover an advantageous embodiment according to which a contract C1, C2, Cn associated with a software application A1, A2, An can be updated or initialized securely. For this purpose, such a contract can be encrypted after any modification and decrypted during the step 110 of a method 100 according to the invention and as shown in FIG. 4. The identifier AID of the software application A1 can moreover include a redundancy code (for example a hash value of said associated software application) so that the step 130 of initializing execution of the application includes processing to verify this redundancy code prior to triggering execution per se of the software application. If the redundancy code calculated corresponds to the value written in the contract, execution of the software application is confirmed and started. Otherwise, said execution is aborted. In this way, the medical imaging platform 4 can verify that the contract is correctly associated with the appropriate version of said software application.

1. Method for integrating and executing a software application, designed to be implemented by a medical imaging platform operating a file system, said method including:
a step of reading a contract dedicated to a software application that generates an item of output data based on an item of input data, said contract consisting of an item of digital content including:
a unique identifier denoting said software application;
an input virtual directory intended to contain a value of the item of input data of the software application;
an output virtual directory intended to contain the value of the item of output data of the software application;
a step of writing the value of the item of input data of the software application to an input directory of the file system;
a step of triggering execution of the software application denoted by the unique identifier with as arguments the access paths to the input directory of the file system and to an output directory of said file system, said access paths being respectively associated with the input and output virtual directories read from said contract;
a step of reading the value of the item of output data of the software application from the output directory of the file system.

2. Method according to claim 1, for which the contract includes a value of an operating parameter of the software application and for which the step of initializing execution of said software application is adapted to include as argument the value of said operating parameter.

3. Method according to claim 1, comprising a step of loading the software application and the contract in a memory of the software platform.

4. Method according to claim 1, for which the software application adopts the form of a computer container, the unique identifier denoting said software application comprised in said contract expressing the image of said computer container.

5. Method according to claim 1, for which the input virtual directory intended to contain the item of input data of the software application is associated in the contract with an attribute specifying the optional or mandatory nature of said item of input data and for which, when such an attribute shows an optional nature of said item of input data, the writing step thereof is implemented only if such an item of input data is available for the medical imaging platform.

6. Method according to claim 1, for which the output virtual directory intended to contain the value of the item of output data of the software application is associated in the contract with a typing attribute of said output and for which the step of reading the value of the item of output data of the software application consists of associating said value of said typing attribute with the value of said item of output data.

7. Method according to claim 1, for which the contract is encoded prior to its integration in the platform and for which the step of reading a contract includes a prior sub-step of decoding said contract.

8. Computer program product including one or more program instructions that can be executed by the processor unit of a computer, said program instructions being capable of being loaded in a non-volatile memory of said computer and the execution of which by said processor unit causes the implementation of a method according to claim 1.

9. Computer-readable storage medium including the instructions of a computer program product according to claim 8.

10. Medical imaging platform comprising a processor unit, a memory used by a file system, said memory comprising the program instructions of a computer program product according to claim 8.
